(19) United States
(12) Patent Application Publication
(10) Pub. No.: US 2018/0119087 A1
(43) Pub. Date: May 3, 2018

(54) SPERM PURIFICATION SYSTEM

(71) Applicants: Chih Peng Chin, Fremont, CA (US); Robert Bailen, University City, MO (US); Huy Lam, San Diego, CA (US); Avik, St. Louis, MO (US)

(72) Inventors: Huy Lam, San Diego, CA (US); Alex Zhang, San Diego, CA (US); Kenny Chin, Fermont, CA (US); Avik Som, St. Louis, MO (US); Blake Marggraff, Lafayette, CA (US); Rishabh Chandak, St. Louis, MO (US); Ege Altan, St. Louis, MO (US)

(21) Appl. No.: 15/569,615

(22) PCT Filed: Apr. 29, 2016
(86) PCT No.: PCT/US2016/030071

§ 371 Date: Oct. 26, 2017

Related U.S. Application Data
(60) Provisional application No. 62/154,513, filed on Apr. 29, 2015.

(51) Int. Cl. C12M 1/00; C12N 5/076

(52) U.S. Cl. CPC C12M 47/04; C12N 5/061

(57) ABSTRACT

A system for purifying sperm for use in an assisted reproductive technique procedure is provided. The system includes a sperm filter through which a chemoattractant and a sperm specimen are pumped. When the chemoattractant is pumped into the filter a gradient is formed of chemoattractants having varying concentrations. Sperm that is desirable will swim toward the higher concentrated chemoattractant and are collected by a sperm collection outlet, while sperm that are immotile and unpreferred are unable to swim across the gradient are collected by a waste collection outlet.

### CROSS-REFERENCE TO RELATED APPLICATIONS

This application claims the benefit of U.S. Provisional Application No. 62/154,513, filed Apr. 29, 2015, which is hereby incorporated by reference in its entirety.

### BACKGROUND OF INVENTION

Currently, the average age for women to get married is 27. This number is rising as more women join and remain in the workforce. Unfortunately, for women who try to become pregnant in their early to mid-thirties, the likelihood of getting pregnant exponentially decreases with age.

In vitro fertilization (IVF) is a long-used medical procedure that assists couples in becoming pregnant. During IVF, mature eggs are retrieved from the ovaries, and the ovaries are fertilized by purified sperm in a laboratory setting. Once an embryo or embryos form, the eggs are placed in the uterus. In 2012, about 165,000 IVF procedures were performed, and this number is expected to grow 10% annually. However, despite its frequent use, IVF has a low rate of success. On average, only 29.4% of attempted cycles for becoming pregnant using IVF lead to pregnancy. Moreover, of those 29.4% that result in pregnancy, only 22.4% result in live births.

A critical step in IVF is the purification of sperm, which involves separating sperm cells from leukocytes, immotile sperm, epithelial cells, and bacteria normally found in a semen sample. Current purification methods jeopardize the success of an IVF cycle. Existing methods use serial centrifugation, which only directly select for sperm based on motility, omitting other critical criteria such as morphology and chemotactic ability. Sperm morphology and chemotaxis have been implicated in numerous studies as important predictors of fertilization and pregnancy (Bartoov et al.; De Vos et al.; Cheung). In addition, centrifugation significantly damages sperm DNA and impairs sperm function through reactive oxygen species damage (Henkel and Schill; Peultier et al.).

The current gold standard to purify sperm, the “swim up” method, exposes sperm to reactive oxygen species which damage sperm DNA and physiology. Therefore, using the swim up method lowers the success rate of IVF. Each cycle of IVF costs approximately $12,400. Therefore, given the flaws in the swim-up method that decrease the likelihood of a successful IVF, multiple cycles can make for a very expensive process.

Alternative methods to the swim up method such as density gradient centrifugation and migration sedimentation only marginally reduce sperm damage for high premiums. When the density gradient centrifugation method is used, the potential for endotoxin poisoning is increased. Also, the washing process requires centrifugal-related DNA integrity damage. The migration sedimentation method is expensive, has a low recovery rate, and is an inordinately complex procedure.

Microfluidic sperm purification technologies previously developed are equally inadequate due to volume limitations (Cho et al.). These technologies also only select for motile sperm alone, omitting other factors such as sperm size and chemotaxis ability. One technology uses sperm chemotaxis to separate two populations of sperm by culturing chemoattractant secreting cells (cumulus cells) in a microfluidic device (patent is listed below (Xie et al.)). However, this prior art doesn't have enough volume to practically purify whole semen samples. In addition, there is not a practical way for sperm retrieval to be used for assisted reproductive technology (ART) procedures. Another technology separates sperm cells by using an electric field. However, all sperm cells (including those that are non-motile and abnormal) are able to be separated in this way. Thus, this technology does not effectively separate viable sperm for ART procedures.

Half of the genetic material of the embryo comes from the sperm, so the sperm's genetic integrity and physiology is a substantial and critical contributing factor for a successful IVF cycle. An alternative method and/or system to the swim-up-method for purifying sperm is needed that does not damage sperm DNA or physiology. In order for a sperm purifying device to be useful, it should effectively separate sperm from the seminal plasma, remove dysfunctional sperm, eliminate leukocytes, and enrich sperm in terms of motility, DNA integrity, and normal morphology. An improved alternative sperm purification method and system that has the above mentioned useful qualities would increase the likelihood of a successful IVF and would reduce the cost of IVF.

### SUMMARY OF THE INVENTION

A device for purifying sperm for subsequent use in ART procedures and/or academic research is provided. The sperm purification system that is subject to the present application seeks to enhance the efficacy and quality of sperm purification by simulating the natural sperm selection process using disposable microfluidics. The sperm purification system hereof not only purifies sperm, but also may select for proper sperm morphology without introducing DNA or reactive oxygen species damage. The system may mimic sperm selection in natural fertilization by using chemical gradients naturally found in the uterus that attract sperm. The system is disposable, and its simple design allows the system to be cost effective and easily adopted.

In a first embodiment, a sperm filter for purifying a sperm specimen for use in an assisted reproductive technology procedure is provided. The sperm filter may include at least one chemoattractant inlet for introducing a chemoattractant into the sperm filter. It also preferably includes at least one sperm inlet for introducing the sperm specimen into the sperm filter. The filter also includes a central channel in fluid communication with both of the inlets, wherein the chemoattractant and the sperm specimen are introduced into the central channel via the inlets.

In the first embodiment, at least one chemoattractant and at least one sperm specimen are introduced into the central channel. A gradient is formed across the central channel with lower chemoattractant concentration near the sperm inlet and greater chemoattractant concentration near the chemoattractant inlet. Both of a waste collection outlet and a sperm collection outlet are provided in fluid communication with the central channel.

The gradient formed in the central channel preferably produces a chemotaxis effect such that chemoactive motile sperm swim across the gradient toward the greater chemoattractant concentration and the sperm collection outlet. Immotile sperm are unable to swim across the gradient and are collected in the waste collection outlet.

In at least one embodiment, the filter also includes a pump in fluid communication with the filter for pumping the at least one chemoattractant and the at least one sperm specimen into the sperm filter. The pump may pump the at least one chemoattractant and the at least one sperm specimen into the sperm filter using a square wave or a different general periodic wave.

In another embodiment, the sperm filter also includes a purification chamber for housing the sperm filter.

In yet another embodiment, the sperm filter may include a sperm selection filter or other sperm morphology selection component that spans across a length of the central channel from the at least one sperm inlet to the sperm collection outlet for preventing sperm having abnormally large heads from transversing the central channel toward a greater concentration of chemoattractant.

The sperm filter may, in one embodiment, include three chemoattractant inlets, each of the three chemoattractant inlets having different chemoattractant concentrations, with the first chemoattractant inlet having the greatest concentration being farthest from the at least one sperm inlet, the second chemoattractant inlet having the lowest concentration being nearest the at least one sperm inlet, and the third chemoattractant inlet being located between the first and second chemoattractant inlets. In that embodiment, the first chemoattractant inlet may be 100 μM progesterone, the second chemoattractant inlet may be 20 μM progesterone, and the third chemoattractant inlet may be 50 μM progesterone.

In the sperm filter, in at least one embodiment, the chemoattractant pumped into the sperm filter is progesterone. In alternative embodiments, follicular fluid/secretions from cumulus cells may be used as an alternative chemoattractant.

In one embodiment, the gradient formed is convex parabolic. In yet another embodiment, the gradient formed is concave parabolic.

In a separate embodiment, a sperm purification system for purifying a sperm specimen for subsequent use in an assisted reproductive technology procedure is provided. The system may include a pump member and a sperm filter in fluid communication with the pump member.

In this embodiment, the sperm filter may include at least one chemoattractant inlet for introducing a chemoattractant into the sperm filter and at least one sperm inlet for introducing the sperm specimen into the sperm filter.

A central channel in fluid communication with the inlets may also be included in the central channel, wherein the chemoattractant and the sperm specimen are introduced into the central channel via the inlets.

In this embodiment, when the pump member is activated, the at least one chemoattractant and the at least one sperm specimen are introduced into the central channel and a gradient is formed across the central channel with lower chemoattractant concentration near the at least one sperm inlet and greater chemoattractant concentration near the at least one chemoattractant inlet.

A waste collection outlet and a sperm collection outlet may each be provided in fluid communication with the central channel. The gradient formed in the central channel produces a chemotaxis effect such that chemoactive motile sperm swim across the gradient to the sperm collection outlet, and immotile sperm fail to swim across the gradient and are collected in the waste collection outlet.

In at least one embodiment of the system, the pump pumps the at least one chemoattractant and the at least one sperm specimen into the sperm filter using a square wave.

In another embodiment of the system, the system also includes a purification chamber in which the sperm filter is housed.

The sperm filter may also include a sperm selection filter that spans across a length of central channel from the at least one sperm inlet to the sperm collection outlet for preventing sperm having abnormally large heads from transversing the central channel toward a greater concentration of chemoattractant.

In a preferred embodiment, there are three chemoattractant inlets, each of the three chemoattractant inlets having different chemoattractant concentrations, with the first chemoattractant inlet having the greatest concentration being farthest from the at least one sperm inlet, the second chemoattractant inlet having the lowest concentration being nearest the at least one sperm inlet, and the third chemoattractant inlet being located between the first and second chemoattractant inlets. In that embodiment, the first chemoattractant inlet may be 100 μM progesterone, the second chemoattractant inlet may be 20 μM progesterone, and the third chemoattractant inlet may be 50 μM progesterone.

In at least one embodiment, the chemoattractant introduced into the sperm filter is progesterone.

In alternative embodiments still, the gradient formed may be convex parabolic or concave parabolic.

The sperm filter may also include a divider between the sperm collection outlet and the waste collection outlet to filter or funnel preferred sperm toward the sperm collection outlet and immotile sperm toward the waste collection outlet.

### BRIEF DESCRIPTION OF THE DRAWINGS

In the accompanying drawings, which form a part of the specification and are to be read in conjunction therewith in which like reference numerals are used to indicate like or similar parts in the various views:

FIG. 1 is a schematic of a sperm purification system arranged and constructed according to the teachings of the present invention;

FIG. 2 is a top plan view of a purification chamber which forms a part of the sperm purification system of FIG. 1;

FIG. 3 is a perspective view of the purification chamber of FIG. 2;

FIG. 4 is a top plan view of a sperm filter for use in the purification chamber of FIGS. 2 and 3;

FIG. 5 is an additional embodiment of the sperm filter of FIG. 4;

FIG. 6 is a graph showing the velocity at which chemoattractants and the semen sample are introduced into a main channel of the sperm filter of FIG. 4;

FIG. 7 is a schematic of a gradient of progesterone generated in the sperm filter of FIG. 4;

FIG. 7a is a graph showing the progesterone concentration across the channel of the sperm filter of FIG. 7;

FIG. 8 is a schematic of a first alternative geometry to generating an alternative gradient of progesterone in the sperm purification system;

FIG. 8a is a graph showing the progesterone concentration across the channel of the sperm filter of FIG. 8;

FIG. 9 is a schematic of a second alternative geometry to generating an alternative gradient of progesterone in the sperm purification system; and

FIG. 9a is a graph showing the progesterone concentration across the channel of the sperm filter of FIG. 9.

### DETAILED DESCRIPTION OF THE INVENTION

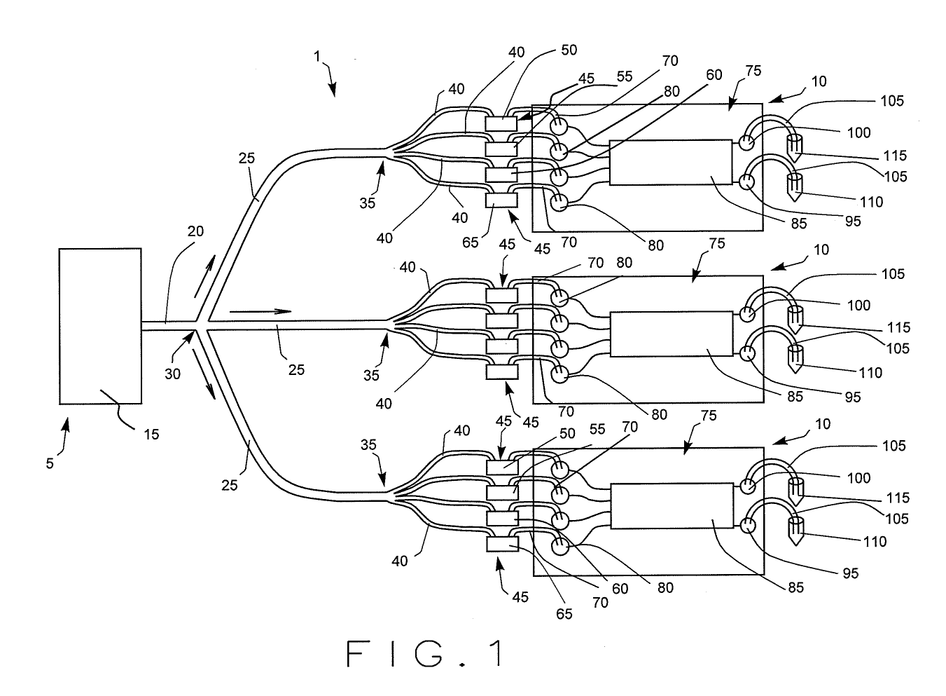

FIG. 1 illustrates a schematic of a sperm purification system 1 according to the teachings of the present invention. Sperm purification system 1 seeks to enhance the efficacy and quality of sperm purification for IVF by simulating the natural sperm selection process using disposable microfluidics, as described in greater detail herein below. Sperm purification system 1 hereof not only purifies sperm, but also may select for proper sperm morphology without introducing DNA or reactive oxygen species damage. System 1 may mimic sperm selection in natural fertilization by using chemical gradients naturally found in the uterus that attract sperm. System 1 is simply designed to be cost effective and easily incorporated by fertility healthcare professionals across the world.

Sperm purification system 1 includes a pumping station 5 for pumping air downstream in a manner described below. Pump 15 may be a piezoelectric pump, syringe pump, or other pump known or foreseeable in the art. In the embodiment illustrated in FIG. 1, pumping station 5 includes a pump member 15 and an outlet tube member 20 which provides an outlet for air to be pumped from pump member 15. Pump member 15 may be of any type able to pump air into fluid stations 10 in a manner consistent to the description below.

First tube members 25 are split from outlet member 20 at a first split location 30 so that multiple fluid stations 10 may be used to increase the efficiency of sperm purification system 1. In an embodiment where a single fluid station 10 is used, a split may not be necessary. Air pressure from pump member 15 is pumped into tube members 25 by way of outlet tube member 20. While FIG. 1 illustrates three tube members 25 splitting from outlet tube member 20 at location 30, in other embodiments more or fewer tube members 25 splitting from outlet tube member 20 are foreseeable.

At a second split location 35 within pumping station 5, tube members 25 each split into four second tube members 40. Tube members 25 are split into four second tube members 40 so that air pressure from pump member 15 is provided to reservoirs or inlets 45. In FIG. 1, four reservoirs 45 are provided, though in alternative embodiments, more or fewer reservoirs 45 may be provided, in a manner consistent with the components and system described below.

Air pressure going into fluid reservoirs 45 feeds fluid into a sperm inlet and low, medium, and high concentration chemoattractant inlets 45 may include a sperm inlet 50, and low, medium, and high concentration chemoattractant inlets 55, 60, and 65, respectively. Sperm samples are preferably introduced into sperm purification system 1 via inlet 50. In the preferred embodiment, inlet 55 provides 20 μM (picomolar) concentrated progesterone, inlet 60 provides 50 μM concentrated progesterone, and inlet 65 provides 100 μM concentrated progesterone. Other foreseeable concentrations of progesterone to which sperm may be attracted may be provided in other embodiments. Also, other chemoattractants may be used in lieu or in combination with progesterone having varying concentrations including, but not limited to, sugar and/or follicular fluid. Inlet tube members 70 are provided at each of inlets 50, 55, 60, 65 to provide fluid communication to fluid station 10.

Fluid station 10 includes a purification chamber 75 (illustrated in greater detail in

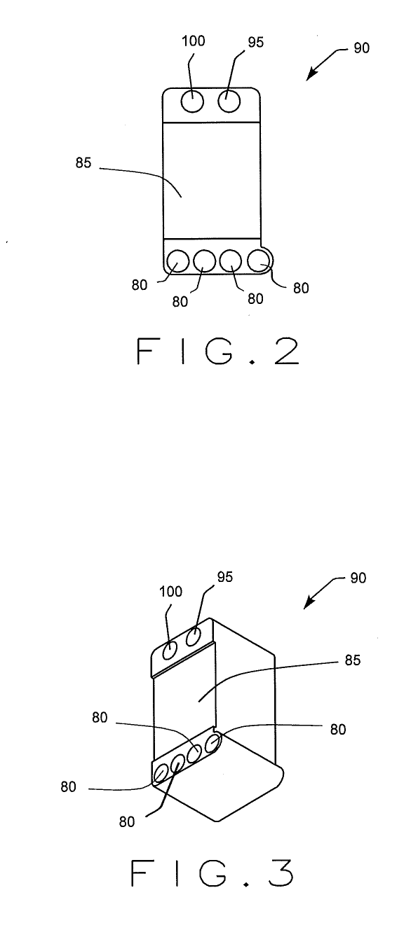

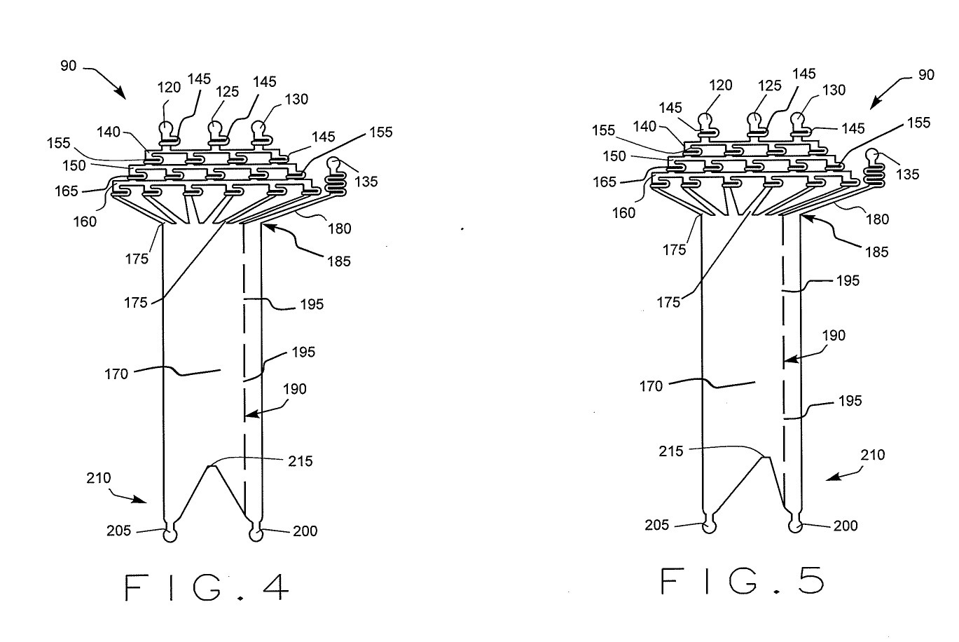

FIGS. 2 and 3) where inlet tube members 70 are preferably received at input sockets 80 of a proximal end of purification chamber 75. Purification chamber 75 also preferably includes a sperm filter socket 85 for receiving and securing sperm filter 90 shown in FIG. 4, and described in detail herein below. A waste socket 95 and a purified sperm socket 100 are preferably provided at a distal end of purification chamber 75. Exit tube members 105 are provided at each of sockets 95, 100, as are collection wells 110, 115. Collection well 110 is provided to collect waste (e.g., unfit sperm specimens), while collection well 115 is provided to collect sperm deemed fertile using the microfluidic selection process described in greater detail herein below. Purification chamber 75 may be made from nearly any material, but in a preferred embodiment is made of an inexpensive material such as acrylonitrile butadiene styrene (ABS) plastic.

In summary, when air pressure is generated by pump member 15, air pressure flows through outlet tube member 20, to tube members 25, to tube members 40. Subsequently air flows into inlets 50, 55, 60, 65 where fluid, either a semen sample or a chemoattractant, is pushed through outlet tube members 70 into chamber 75. The sperm selection process described below may then take place, and the fluid pressure from reservoir 45 also pushes waste and purified sperm through exit tube members 105 and into wells 110, 115. Sperm deemed fertile collected in collection well 115 may then be used in IVF or other fertilization procedures in manners known and understood by healthcare professionals.

When a semen sample is provided, it is introduced at sperm inlet 50, while chemoattractants are introduced at one or more of inlets 55, 60, 65. The semen and chemoattractants are preferably introduced into sperm filter 90 shown in FIG. 4 by way of input sockets 80. Sockets 80 are best illustrated in FIGS. 1-3. Input sockets 80 are preferably in fluid communication with sperm filter 90 when sperm filter 90 is placed within filter socket 85 via chemoattractant inlets 120, 125, 130 and sperm inlet 135. Preferably, chemoattractant inlets 120, 125, 130, and sperm inlet 135 are in fluid communication with inlets 55, 60, 65, and 50, respectively.

When pump member 15 is active, each of inlets 120, 125, 130, flows a chemoattractant into a first channel 140 by way of tube members 145, then into a second channel 150 by way of tube members 155, then into a third channel 160 by way of tube members 165. Chemoattractant is subsequently flowed into a main central channel 170 of sperm filter 90 by way of tube members 175. Similarly, when pump member 15 is active, sperm inlet 135 flows semen sample into central channel 170 by way of a tube member 180.

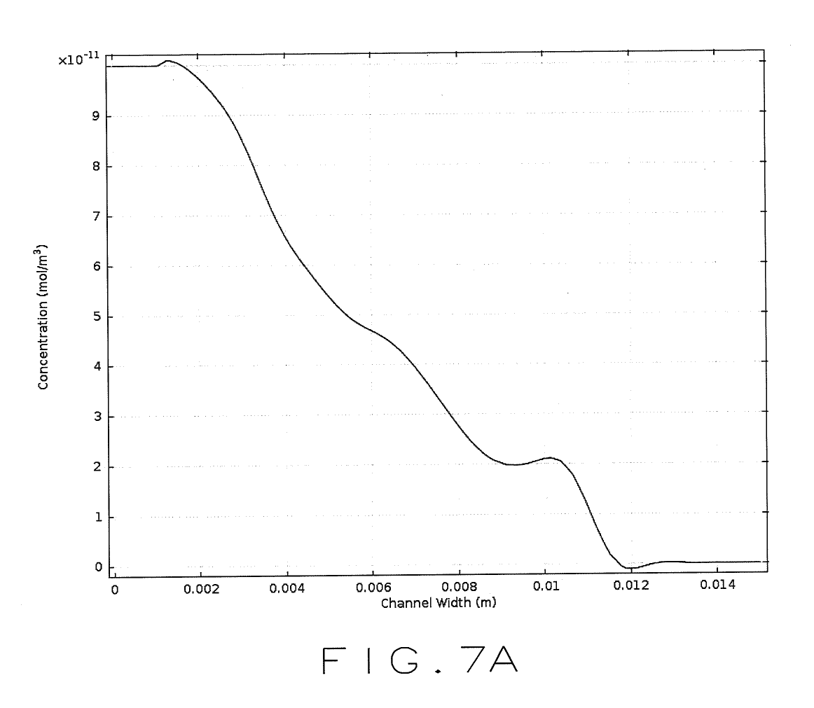

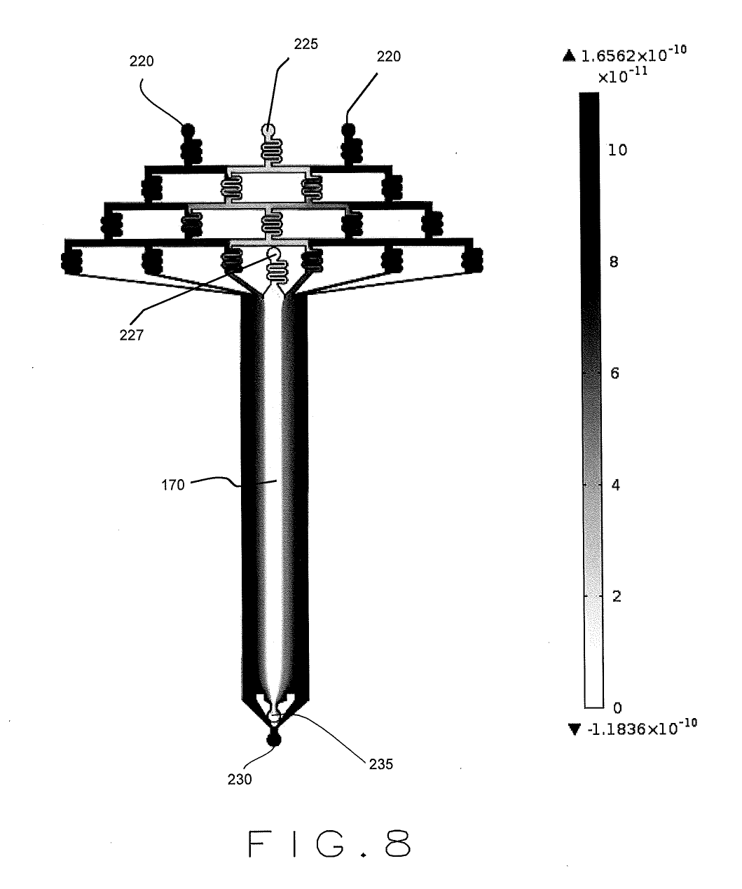

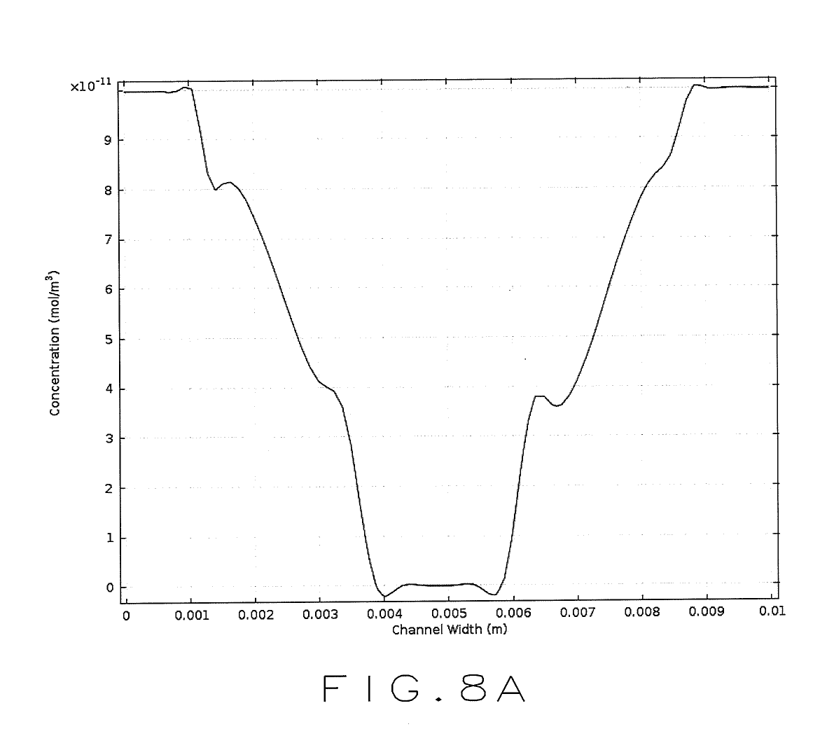

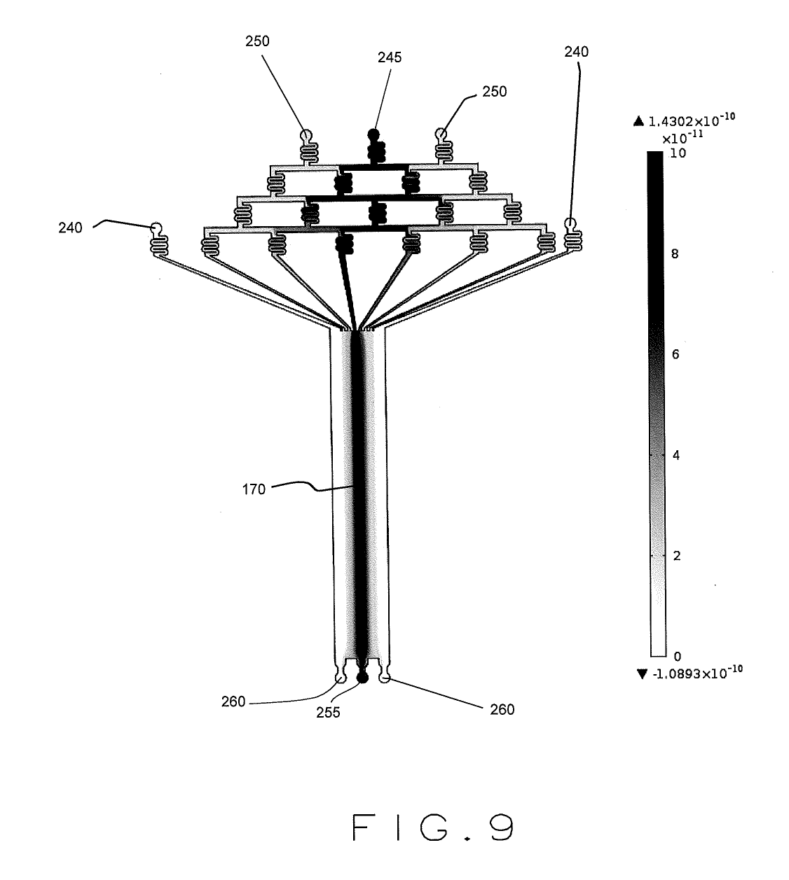

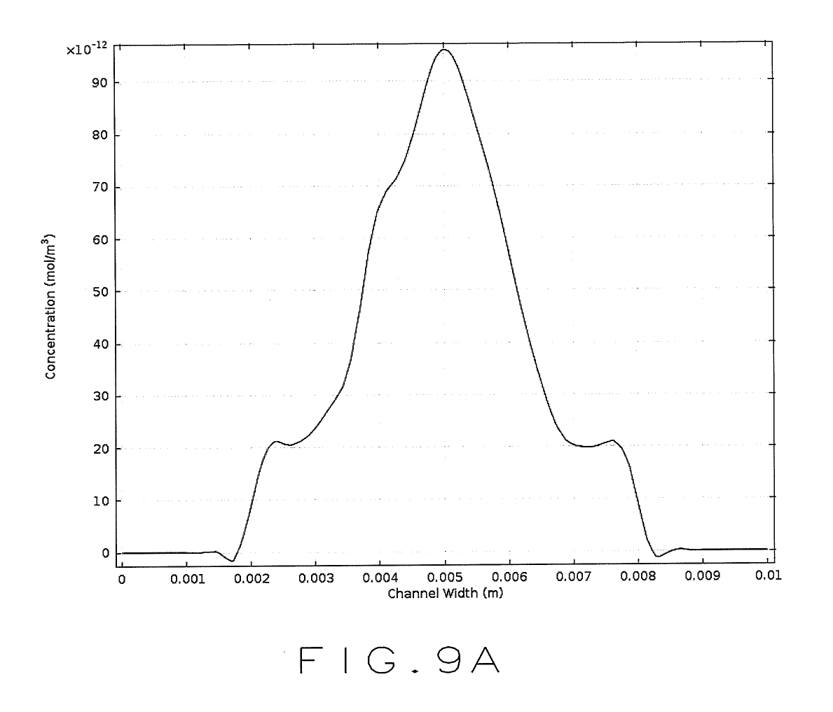

Tube members 145, 155, 165, 175 may be S-shaped as shown in the embodiment illustrated in FIG. 4. Tube members 145, 155, 165, 175 being S-shaped may improve the flow rate of fluids traveling therethrough, and may further help prevent bubbles from forming in the tube members 145, 155, 165, 175. In alternative embodiments not illustrated, tube members 145, 155, 165, 175 may be C-shaped, cylindrical, or other alternative shapes known or foreseeable to those skilled in the art of fluid transport. Channels 140, 150, 160 may be used to help form a gradient in central channel 170, such as the gradient shown in FIG. 7, a description of which is provided below. Together tube members 145, 155, 165, 175 and channels 140, 150, 160 form a tree-like structure that could appear differently in different embodiments if more or fewer tube members and/or channels were provided, for example in the embodiments shown in FIGS. 8 and 9 described below. As the number of tube members 145, 155, 165, 175 and/or channels 140, 150, 160 increases, the chemoattractant gradient generated may be made smoother.

Tube members 175, 180 empty into central channel 170 at a first proximal end 185 of central channel 170. Semen samples that have traveled through tube member 180 may not be instantaneously introduced into contact with chemoattractants from tube members 175 because a sperm selection filter 190 including a plurality of channels 195 may be placed within central channel 170. To select for the sperm head size and exclude sperm with abnormally large head sizes (over 8 μm), channels 195 are preferably 10 μm wide, spaced 15 μm apart, and span the length of sperm filter 90.

A waste outlet 200 and a collection outlet 205 are provided at a distal end 210 of central channel 170. Waste outlet 200 and collection outlet 205 are preferably in fluid communication with waste socket 95 and sperm socket 100. A divider 215 may separate waste outlet 200 and collection outlet 205 from one another. Divider 215 may be used to optimize the yield of device 1 and facilitate the pumping of sperm toward outlets 200, 205 of the device. Divider 215 can be moved across the width of the purification chamber to increase or decrease yield at the sacrifice of purity, as shown in FIG. 5. More than one divider may also be implemented depending on the progesterone gradient profile. FIG. 5 shows how one divider can be moved when the sperm are under the influence of a linear concentration gradient.

Sperm filter 90 may be made of polydimethylsiloxane (PDMS), which is an inexpensive, disposable, flexible, and lightweight polymer that is plasma bonded to glass. However, in other embodiments, other polymers can be used such as polymethyl methacrylate (PMMA), cyclic olefin copolymer (COC), cyclic olefin polymers (COP), and polystyrene (PS). These polymers have been shown to be chemically inert, and studies have shown that sperm are not damaged when exposed to the polymers. Any material may be used to construct sperm filter 90 so long as it does not damage sperm when exposed to the polymers.

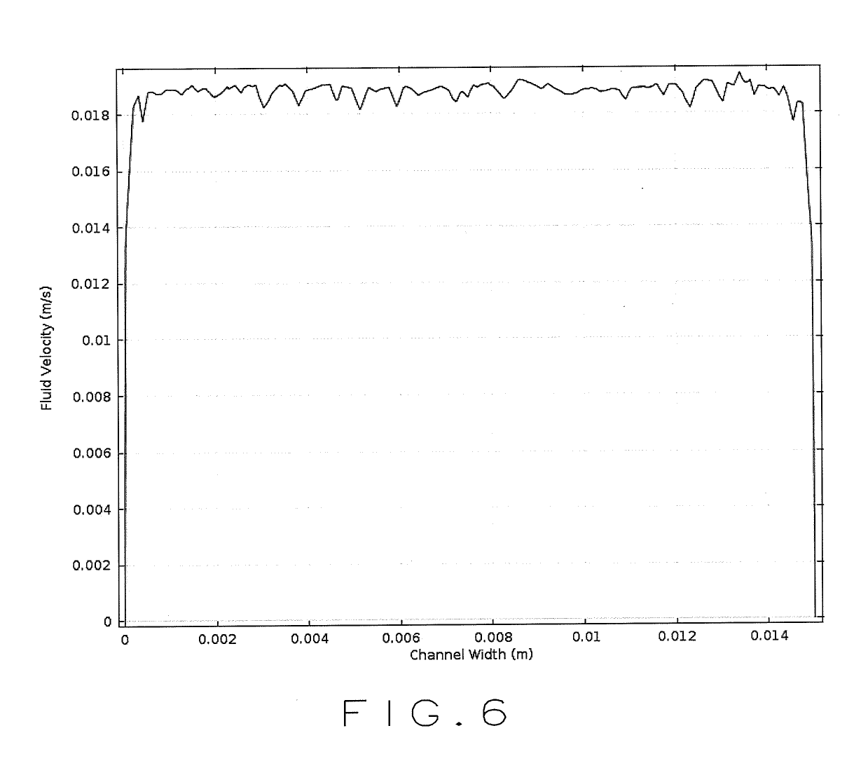

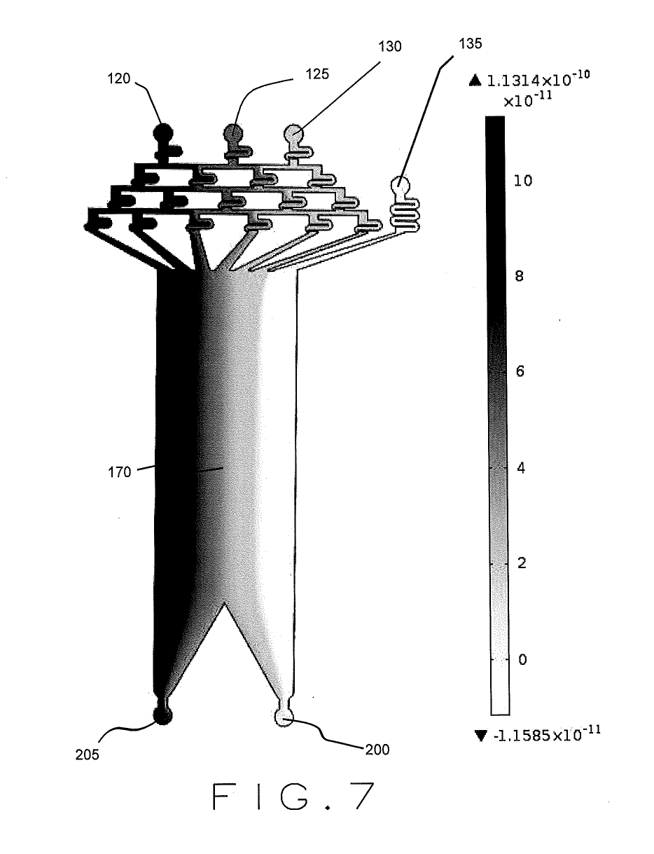

When pump member 15 is active, pump member 15 preferably pumps progesterone from inlets 55, 60, 65 and sperm from sperm inlet 50 into central channel 170. Air pressure produced by pump member 15 preferably generates a square pressure wave (not illustrated). In the preferred embodiment, the square wave has a minimum pressure of 13 mbar and a maximum pressure of 63 mbar, a period of 30 seconds, and a dissymmetry that only creates a 1 second pulse. This pressure profile allows the device to fill under laminar flow with a fluid velocity of ˜0.018 m/s at the peak of the pulse and 0 m/s at the trough of the pulse, as shown in the line graph provided in FIG. 5. This exemplifies a velocity slow enough for sperm to swim against or through could the gradient also be used. The graph shown in FIG. 6 shows fluid velocity across central channel 170 in an embodiment where central channel 170 is 10 mm wide. Other widths for central channel 170 are certainly envisioned, and the graph in FIG. 6 represents a non-limiting example only. In alternative embodiments, other pressures that effectively deliver sperm samples and progesterone or other chemoattractants may be utilized.

The geometry of sperm filter 90 illustrated in FIG. 4 may produce the laminar progesterone gradient illustrated in FIG. 7 and graphed in FIG. 7a when inlet 55 provides 20 μM concentrated progesterone, inlet 60 provides 50 μM concentrated progesterone, and inlet 65 provides 100 μM concentrated progesterone, and the pressure wave velocities modeled in the graph shown in FIG. 6 is output by pump member 15. In the representations of FIGS. 7, 7A, central channel 170 is 15 mm, but it could be wider or narrower in alternative embodiments. As shown in FIGS. 7, 7A, concentration of progesterone is greater near the top of central channel 170 closer to where inlet 65 provides the higher concentrated 100 μM progesterone. Progesterone concentration is lower near the bottom of central channel 170 closer to where sperm inlet 50 provides the semen sample and inlet 55 provides the lower concentrated 20 μM progesterone.

By generating a gradient across sperm filter 90 such as the gradient illustrated in FIG. 7, a chemotaxis effect is generated, and healthy sperm released into central channel 170 that transverse selection filter 190 via channels 195 are able to sense and swim from right to left towards higher concentrations of progesterone. The downward fluid flow due to the square pressure pulse pushes the most viable sperm for assisted reproductive technology such as IVF procedures on the left side of the device towards the sperm socket 100 and sperm collection well 115, while the less viable sperm fails to cross the gradient and fails to swim from right to left toward higher concentrated progesterone levels. These sperm which may be malformed, bad swimmers, or too large to transverse selection filter, are passed toward waste socket 95 and waste collection well 110.

The profile of the progesterone gradient such as the gradient of FIG. 7 may also be changed by slightly changing the geometry and changing the progesterone series dilution. A chemoattractant gradient may even be formed by only introducing a single chemoattractant and a single sperm specimen into central channel 170.

Sperm purification can take place under a convex parabolic progesterone gradient if the sperm inlet is placed in the center of the device instead of the right side, as shown in the alternative gradient modeled in FIG. 8 and the graph provided in FIG. 8A that shows the progesterone concentration across the width of channel 170. In the gradient generated in FIGS. 8, 8A the progesterone dilution series is altered by pumping 100 μM progesterone in outer inlets 220 (20 μM progesterone in inlet 225. Sperm is pumped in inlet 227 above central channel 170). In this embodiment, the sperm that is pumped into the middle of the purification chamber swim outwards towards the sides that have high concentrations of progesterone. The fluid flow then pushes the sperm down to be collected. The collection well in this embodiment, collection well 230 is now the further most outlet, and the waste outlet, waste outlet 235 is the inner most outlet.

As shown in the gradient generated in FIG. 9 and graph of FIG. 9A, sperm purification can take place under a concave parabolic progesterone gradient if sperm inlets 240 are placed on both sides of central channel 170. The progesterone dilution series is altered by pumping 100 μM progesterone in middle progesterone inlet 245 and 10 μM progesterone in the progesterone inlets 250 on either side of inlet 245. In this iteration, the sperm that is pumped into both sides of the purification chamber via sperm inlets 240 and swim towards the center of central channel 170 that has high concentrations of progesterone. The fluid flow then pushes the sperm down to be collected. Collection well 255 is now the center outlet, and waste outlets 260 are provided at both edges of central channel 170.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention for aiding in the purification of sperm samples for ART fertility procedures or studies. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”.

Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

### APPENDIX

### Relevant Patents

U.S. Pat. No.: 0,199,720 A1, Qui et al.¹⁴

The invention provides a microfluidic device that can be used for sperm motility classification, The patent claims include using an electrical gradient to attract sperm, applying an external force to move sperm, and adding a camera to measure motility of sperm cells. The sperm classified as motile can then be used for in vitro fertilization.

U.S. Pat. No.: 6,398,719 B1, Kaneko et al.¹⁷

This is a patent describing a particular sperm washing and concentration method. It is of relevance to our project since sperm concentration is one of the proposed steps in our design scope. The patent claims include a new method for sperm washing using a glass tube with two portions—top and bottom, separated by an elastic bladder. The top portion is filled with a density gradient carrier. The sperm sample is mixed with HANKS solution and then added onto the top portion and the tube is centrifuged. The sediment collected in the bottom portion at the end of the centrifugation contains sperm cells and is used, and everything else is disposed of The density gradient carrier is either colloidal silica or polymerized sucrose.

U.S. Pat. No.: 8,123,924 B2, Aitken et al.¹⁸

The patent claims include a process for separating a sperm type from a sperm population in a sperm sample by electrophoresis comprising subjecting the sperm population to an electric potential such that a sperm type moves through an ion-permeable barrier and the sperm type is separated from the sperm population through the ion-permeable barrier. The sperm type can have any desired characteristics such as motility, robustness, fertilization potential, and a combination thereof.

### US Patent Application: US 2013/0244270 A1 Xie et. al

The present invention relates to a microfluidic channels and chambers. Cells which can secret a chemoattractant or chemorepellent are selectively planted in the gradient can be established in the channels.

https://patents.google.com/patent/US20130244270A1/en?q=chemical&q=gradient&q=microfluidic&q=sperm

### REFERENCES

Bartoov, B., et al. “Pregnancy Rates Are Higher with Intracytoplasmic Morphologically Selected Sperm Injection Than with Conventional Intracytoplasmic Injection.” Fertility and Sterility 80.6 (2003): 1413-19. Print.

### Cheung, Anthony P. “The Significance of Sperm Morphology.” (2015). Print.

### Cho, B. S., et al. “Passively Driven Integrated Microfluidic System for Separation of Motile Sperm.” Anal Chem 75.7 (2003): 1671-5. Print.

De Vos, A., et al. “Influence of individual Sperm Morphology on Fertilization, Embryo Morphology, and Pregnancy Outcome of Intracytoplasmic Sperm Injection.” Fertility and Sterility 79.1 (2003): 42-48. Print.

### Henkel, R. R., and W. B. Schill. “Sperm Preparation for Art.” Reprod Biol Endocrinol 1 (2003): 108. Print.

### Peultier, A. S., et al. “[Fertilization Failure in Ivf and Icsi].” J Gynecol Obstet Biol Reprod (Paris) 44.4 (2015): 380-6. Print.

Wu, J. M., et al. “A Surface-Modified Sperm Sorting Device with Long-Term Stability.” Biomed Microdevices 8.2 (2006): 99-107. Print.
Xie, L., et al. “Integration of Sperm Motility and Chemotaxis Screening with a Microchannel-Based Device.” Clin Chem 56.8 (2010): 1270-8. Print.

What is claimed is:

1. A sperm filter for purifying a sperm specimen for use in an assisted reproductive technology procedure, the sperm filter comprising:
at least one chemoattractant inlet for introducing a chemoattractant into the sperm filter;
at least one sperm inlet for introducing the sperm specimen into the sperm filter;
a central channel in fluid communication with the at least one chemoattractant inlet and the at least one sperm inlet, wherein the chemoattractant and the sperm specimen are introduced into the central channel via the at least one chemoattractant inlet and the at least one sperm inlet, respectively;
wherein when the at least one chemoattractant and the at least one sperm specimen are introduced into the central channel, a gradient is formed across the central channel with lower chemoattractant concentration near the at least one sperm inlet and greater chemoattractant concentration near the at least one chemoattractant inlet;
a waste collection outlet in fluid communication with the central channel;
a sperm collection outlet in fluid communication with the central channel; and
wherein the gradient formed in the central channel produces a chemotaxis effect such that chemoactive motile sperm swim across the gradient toward the greater chemoattractant concentration to the sperm collection outlet, and immotile sperm fail to swim across the gradient and are collected in the waste collection outlet.

2. The sperm filter of claim 1, wherein the filter also includes a pump in fluid communication with the filter for pumping the at least one chemoattractant and the at least one sperm specimen into the sperm filter.

3. The sperm filter of claim 2, wherein the pump pumps the at least one chemoattractant and the at least one sperm specimen into the sperm filter using a square wave.

4. The sperm filter of claim 1, wherein the sperm filter also includes a purification chamber in which the sperm filter is housed.

5. The sperm filter of claim 1, wherein the sperm filter includes a sperm selection filter that spans across a length of the central channel from the at least one sperm inlet to the sperm collection outlet for preventing sperm having abnormally large heads from transversing the central channel toward a greater concentration of chemoattractant.

6. The sperm filter of claim 1, wherein the sperm filter includes three chemoattractant inlets, each of the three chemoattractant inlets having different chemoattractant concentrations, with the first chemoattractant inlet having the greatest concentration being farthest from the at least one sperm inlet, the second chemoattractant inlet having the lowest concentration being nearest the at least one sperm inlet, and the third chemoattractant inlet being located between the first and second chemoattractant inlets.

7. The sperm filter of claim 6, wherein the first chemoattractant inlet is 100 μM progesterone, the second chemoattractant inlet is 20 μM progesterone, and the third chemoattractant inlet is 50 μM progesterone.

8. The sperm filter of claim 1, wherein the at least one chemoattractant is progesterone.

9. The sperm filter of claim 1, wherein the gradient formed is convex parabolic.

10. The sperm filter or claim 1, wherein the gradient formed is concave parabolic.

11. A sperm purification system for purifying a sperm specimen for subsequent use in an assisted reproductive technology procedure, the system comprising:
a pump member;
a sperm filter in fluid communication with the pump member, the sperm filter including:
at least one chemoattractant inlet for introducing a chemoattractant into the sperm filter;
at least one sperm inlet for introducing the sperm specimen into the sperm filter;
a central channel in fluid communication with the at least one chemoattractant inlet and the at least one sperm inlet, wherein the chemoattractant and the sperm specimen are introduced into the central channel via the at least one chemoattractant inlet and the at least one sperm inlet, respectively;
wherein when the pump member is activated, the at least one chemoattractant and the at least one sperm specimen are introduced into the central channel and a gradient is formed across the central channel with lower chemoattractant concentration near the at least one sperm inlet and greater chemoattractant concentration near the at least one chemoattractant inlet;
a waste collection outlet in fluid communication with the central channel; and
a sperm collection outlet in fluid communication with the central channel; and
wherein the gradient formed in the central channel produces a chemotaxis effect such that chemoactive motile sperm swim across the gradient to the sperm collection outlet, and immotile sperm fail to swim across the gradient and are collected in the waste collection outlet.

12. The sperm purification system of claim 11, wherein the pump pumps the at least one chemoattractant and the at least one sperm specimen into the sperm filter using a square wave.

13. The sperm purification system of claim 11, wherein the sperm purification system also includes a purification chamber in which the sperm filter is housed.

14. The sperm purification system of claim 11, wherein the sperm filter includes a sperm selection filter that spans across a length of the central channel from the at least one sperm inlet to the sperm collection outlet for preventing sperm having abnormally large heads from transversing the central channel toward a greater concentration of chemoattractant.

15. The sperm purification system of claim 11, wherein the sperm filter includes three chemoattractant inlets, each of the three chemoattractant inlets having different chemoattractant concentrations, with the first chemoattractant inlet having the greatest concentration being farthest from the at least one sperm inlet, the second chemoattractant inlet having the lowest concentration being nearest the at least one sperm inlet, and the third chemoattractant inlet being located between the first and second chemoattractant inlets.

16. The sperm purification system of claim 15, wherein the first chemoattractant inlet is 100 μM progesterone, the second chemoattractant inlet is 20 μM progesterone, and the third chemoattractant inlet is 50 μM progesterone.

17. The sperm purification system of claim 11, wherein the at least one chemoattractant is progesterone.

18. The sperm purification system of claim 11, wherein the gradient formed is convex parabolic.

19. The sperm purification system of claim 11, wherein the gradient formed is concave parabolic.

20. The sperm purification system of claim 11, wherein the sperm filter also includes a divider between the sperm collection outlet and the waste collection outlet.
